(19) United States
(12) Patent Application Publication
(10) Pub. No.: US 2019/0298882 A1
(43) Pub. Date: Oct. 3, 2019

(54) HYDROGEL BANDAGE

(71) Applicant: Kevin M. Nelson, Akron, OH (US)

(72) Inventor: Kevin M. Nelson, Akron, OH (US)

(21) Appl. No.: 15/936,630

(22) Filed: Mar. 27, 2018

(51) Int. Cl. A61L 15/60; A61L 15/26; A61L 15/58; A61L 15/24; A61L 15/20; A61L 15/22; A61F 13/02

(52) U.S. Cl. CPC A61L 15/60; A61F 2013/00293; A61L 15/58; A61L 15/24; A61L 15/20; A61L 15/225; A61F 13/0253; A61F 13/0256; A61F 13/0289; A61F 13/0213; A61F 13/0259; A61F 13/0223; A61F 13/022; A61L 2420/02; A61L 2420/08; A61L 15/26

(57) ABSTRACT

A bandage including a padding material, a hydrogel material; and a substrate is taught. The padding material of the bandage is co-disposed by the hydrogel material and both the padding material and the hydrogel material are carried by the substrate. The bandage is created by providing a substrate having a first and second planar surface. Then, a hydrogel material is placed onto the second planar surface of the substrate. Finally, a padding material is placed onto the second planar surface of the substrate so that the padding material is co-disposed by the hydrogel material.

### FIELD OF THE INVENTION

The present invention relates to a medical device which employs a hydrogel-based bandage for treatment of wounds, burns, cuts, tears, blisters, ulcers and the like on the human body and a method for forming the same. More particularly, this invention relates to a bandage comprising a hydrogel, an absorbent padding material, and a substrate to which the hydrogel and padding material are adhered.

### BACKGROUND OF THE INVENTION

A wide variety of materials have been used to treat wounds using different types of dressings. Typical materials that are used in dressings include individual wound contact materials, absorbent materials, adhesive binding and non-adhesive binding materials and fastening materials. Dressings are also available that integrate these components and are manufactured as a single item. These range from small dressings such as adhesive bandages all the way up to larger adhesive dressings.

A bandage can prevent contamination, provide compression to minimize swelling and hold topical medications against the wound. However, most bandages on the market contain adhesives that can cause reactions to those people having sensitive skin or to those people having allergic reactions to certain adhesives. Although there are bandages available that claim to be hypoallergenic and bandages available that claim to be useable by those with sensitive skin; there are no bandages on the market that prove to be effective or suitable for those with the need for a hypoallergenic bandage and a need for a bandage useable for those with sensitive skin. Therefore, presently there is no solution for those with sensitive skin who also need hypoallergenic adhesive bandages.

U.S. Pat. No. 4,909,244 issued to Quarfoot et al. relates to integral, composite wound dressings employing a hydrogel layer for absorbing and retaining exudate as well as an outer layer which protects the dressing and the wound while at the same time being vapor-permeable to permit transpiration of liquid and thus provide maximal protection against pooling.

U.S. Pat. No. 5,160,328 issued to Cartmell et al. relates to a bandage containing a polyurethane hydrogel material suitable for absorbing bodily fluids such as wound exudate without adhering to a wound on which the bandage is mounted.

U.S. Pat. No. 5,674,346 issued to Kundel relates to novel processes for adhering polymeric hydrogels to an adhesive coated surface of a substrate and to novel hydrogel laminates and bandages and methods for forming the same.

U.S. Pat. No. 6,861,067 B2 issued to McGhee et al. relates to a hydrogel wound dressing and a method of making and using the same. More particularly, McGhee relates to a flexible hydrogel wound dressing which is highly absorptive, contours to a wound site and maintains a wound in a moist state to promote healing thereof and the method of producing and using the same.

U.S. Patent Application Publication 2013/0072843 A1 to Huang relates to the field of medical dressing technology, specifically providing a dressing that can keep a suitable environment for wound healing, shortens the time of wound healing, and reduces the chances of the wound becoming infected.

U.S. Pat. No. 4,871,490 issued to Rosiak et al. relates to a method of manufacturing dressings in the form of biological gels, involving making a water suspension of gelatin, a natural polymer additionally containing pectins, bactericidal substances, salts of divalent iron and, possibly, carboxymethylcellulose and polyisobutylene and subjected to sterilization.

It is an object of this invention to provide hydrogel bandages with improved wearing properties as to provide a higher quality of care for the user's skin. It is a further object of this invention to provide a process by which a hydrogel and padding material can be co-disposed to an adhesive coated surface of a substrate.

### SUMMARY OF THE INVENTION

In a first embodiment, the present invention provides a bandage comprising: a padding material, a hydrogel material, and a substrate; wherein the padding material is co-disposed with the hydrogel material and wherein both the padding material and the hydrogel material are carried by the substrate.

In a second embodiment, the present invention provides a bandage as in the first embodiment, wherein the substrate has a first planar surface and a second planar surface and wherein the second planar surface is coated with an adhesive polymer.

In a third embodiment, the present invention provides a bandage as in either the first or second embodiment, wherein the padding material and the hydrogel material are carried by the substrate by means of the adhesive polymer coated on the second planar surface of the substrate.

In a fourth embodiment, the present invention provides a bandage as in any of the first through third embodiments, wherein the padding material comprises a first planar surface and a second planar surface, wherein the hydrogel material comprises a first planar surface and as second planar surface, and wherein both the first planar surface of the hydrogel material and the first planar surface of the padding material are placed on the second planar surface of substrate.

In a fifth embodiment, the present invention provides a bandage as in any of the first through fourth embodiments, wherein the substrate is a polyurethane film.

In a sixth embodiment, the present invention provides a bandage as in any of the first through fifth embodiments, wherein the padding material is selected from the group consisting of a hydrocolloid, cotton gauze, foam, a blend of lyocell and polyester, and combinations thereof.

In a seventh embodiment, the present invention provides a bandage as in any of the first through sixth embodiments, wherein the hydrogel material is selected from the group consisting of co-polyacrylate/acrylamide, glycerin, n-methyldiethanolamine, triethanolamine, potassium chloride, fumed silica, polyvinylpyrrolidone, water, and combinations thereof.

In an eighth embodiment, the present invention provides a bandage as in any of the first through seventh embodiments, wherein the hydrogel material has a stainless-steel adhesion of from about 100 g/in² to about 2,000 g/in² and a pH of from about 4.0 to about 8.0.

In a ninth embodiment, the present invention provides a bandage as in any of the first through eighth embodiments, wherein the hydrogel material has a water absorption capacity of from about 25% to about 3,500% and a thickness of from about 5 mils to about 70 mils.

In a tenth embodiment, the present invention provides a bandage as in any of the first through ninth embodiments, further comprising a release liner adhered to the bandage at a position opposite a position of the substrate.

In an eleventh embodiment, the present invention provides a process for creating a bandage comprising the steps of providing a substrate having a first planar surface and a second planar surface; placing a hydrogel material onto the second planar surface of the substrate; and placing a padding material onto the second planar surface of the substrate such that the padding material is co-disposed by the hydrogel material.

In a twelfth embodiment, the present invention provides a process for creating a bandage as in the eleventh embodiment wherein the second planar surface of the substrate is coated with an adhesive polymer.

In a thirteenth embodiment, the present invention provides a process for creating a bandage as in any of the eleventh through twelfth embodiments, wherein the substrate comprises a polyurethane film.

In a fourteenth embodiment, the present invention provides a process for creating a bandage as in any of the eleventh through thirteenth embodiments, wherein the padding material is selected from the group consisting of a hydrocolloid, cotton gauze, foam, a blend of lyocell and polyester, and combinations thereof.

In a fifteenth embodiment, the present invention provides a process for creating a bandage as in any of the eleventh through fourteenth embodiments, wherein the hydrogel material is selected from the group consisting of co-polyacrylate/acrylamide, glycerin, n-methyldiethanolamine, triethanolamine, potassium chloride, fumed silica, polyvinylpyrrolidone, water, and combinations thereof.

In a sixteenth embodiment, the present invention provides a process for creating a bandage as in any of the eleventh through fifteenth embodiments, wherein the hydrogel material has a stainless-steel adhesion of from about 100 g/in² to about 2,000 g/in² and a pH of from about 4.0 to about 8.0.

In a seventeenth embodiment, the present invention provides a process for creating a bandage as in any of the eleventh through sixteenth embodiments, wherein the hydrogel material has a water absorption of from about 25% to about 3,500% and a thickness of about 5 mils to about 70 mils.

In an eighteenth embodiment, the present invention provides a process for creating a bandage as in any of the eleventh through seventeenth embodiments, further comprising the step of placing a release liner on the bandage at a position opposite the substrate.

### BRIEF DESCRIPTION OF THE DRAWINGS

FIG. 1 is a bottom view of an embodiment of the present invention comprising a rectangular island pad style bandage;

FIG. 2 is a bottom perspective view of an embodiment of the present invention comprising a rectangular island pad style bandage;

FIG. 3 is a top perspective view of an embodiment of the present invention comprising a rectangular island pad style bandage;

FIG. 4 is a bottom view of an embodiment of the present invention comprising a traditional style bandage; and

FIG. 5 is an exploded perspective view of an embodiment of the present invention comprising a circular island pad style bandage.

### DETAILED DESCRIPTION OF ILLUSTRATIVE EMBODIMENTS

The present invention relates to a medical device that employs a hydrogel-based bandage that functions as a wound bandage that adheres effectively to the skin. The present invention uses a hydrogel as the adhering/adhesive component, instead of commonly used adhesives in bandages, to offer an alternative form of adhering bandages. As compared to the adhesives commonly used with more bandages, the use of hydrogel is less of a skin irritant for those with sensitive skin, improves protection of a wound from bacterial invasion, and improves moisture of the skin surrounding the wound, also known as the periwound skin.

In one or more embodiments of the present invention, a bandage is provided that comprises a hydrogel and a padding material, which functions as the protectant of the wound to be healed. The padding material also functions to absorb any blood or exudate discharged from the wound.

In one or more embodiments of the present invention, the hydrogel used is biocompatible with human use. The hydrogel used may exist in its original form as an amorphous hydrogel or a sheet hydrogel. In one or more embodiments of the present invention, the hydrogel used in the present invention has a water absorption capacity of at least 25%, in other embodiments at least 50%, and in yet other embodiments at least 100%. In one or more embodiments of the present invention, the hydrogel has a water absorption capacity of at most about 3,500%, in other embodiments at most about 3,300%, and in yet other embodiments at most about 3,000%. In one or more embodiments of the present invention the hydrogel has a water absorption capacity of from about 25% to about 3,500%, in other embodiments from about 50% to about 3,300%, and in yet other embodiments from about 100% to about 3,000%.

In one or more embodiments of the present invention, the hydrogel has a stainless-steel adhesion of at least about 100 g/in², in other embodiments at least about 120 g/in², and in yet other embodiments at least about 135 g/in². In one or more embodiments of the present invention, the hydrogel has a stainless-steel adhesion of at most about 2,000 g/in², in other embodiments at most about 1,800 g/in², and in yet other embodiments at most about 1,600 g/in². In one or more embodiments of the present invention the hydrogel has a stainless-steel adhesion of from about 100 g/in² to about 2,000 g/in², in other embodiments from about 120 g/in² to about 1,800 g/in², and in yet other embodiments from about 135 g/in² to about 1,600 g/in².

A hydrogel with a stainless-steel adhesion within the ranges mentioned above is important in use of the present invention due to a hydrogel with such a stainless-steel adhesion exhibiting an effective adhesion property and easy removal after being applied to the human skin. A hydrogel having these characteristics also allows for the bandage of the present invention to be removed and reapplied multiple times during use without hindering the adhesive properties of the hydrogel.

In one or more embodiments of the present invention, the hydrogel has a pH of at least about 4.0, in other embodiments at least about 4.5, and in yet other embodiments at least about 4.7. In one or more embodiments of the present invention, the hydrogel has a pH of at most about 8.0, in other embodiments at most about 7.5, and in yet other embodiment at most about 7.0. In one or more embodiments of the present invention, the hydrogel has a pH of from about 4.0 to about 8.0, in other embodiments of from about 4.5 to about 7.5, and in yet other embodiments of from about. 4.7 to about 7.0.

In one or more embodiments of the present invention, the hydrogel has a thickness of at least about 5 mils, in other embodiments of at least about 10 mils, and in yet other embodiments at least about 15 mils. In one or more embodiments of the present invention, the hydrogel has a thickness of at most about 70 mils, in other embodiments at most about 60 mils, and in yet other embodiments at most about 45 mils. In one or more embodiments of the present invention, the hydrogel has a thickness of from about 5 mils to about 70 mils, in other embodiments of from about 10 mils to about 60 mils, and in yet other embodiments of from about 15 mils to about 45 mils.

A bandage having a hydrogel having a thickness within the ranges mentioned above is important in use of the present invention due to its better flexibility and allowing the hydrogel to not be so thick as to increase the risk of the bandage of the present invention being caught on the user's garments and being accidently removed.

In one or more embodiments of the present invention, the hydrogel has a volume resistivity of at least about 25 Ohm-in, in other embodiments at least about 35 Ohm-in, and in yet other embodiments at least about 45 Ohm-in. In one or more embodiments of the present invention, the hydrogel has a volume resistivity of at most about 2,500 Ohm-in, in other embodiments at most about 2,400 Ohm-in, and in yet other embodiments at most about 2,300 Ohm-in. In one or more embodiments of the present invention the hydrogel has a volume resistivity of from about 25 Ohm-in to about 2,500 Ohm-in, in other embodiments from about 35 Ohm-in to about 2,400 Ohm-in, and in yet other embodiments from about 45 Ohm-in to about 2,300 Ohm-in.

In one or more embodiments of the present invention, the hydrogel has a peak tack of at least about 15 grams, in other embodiments at least about 25 grams, and in yet other embodiments at least about 40 grams. In one or more embodiments of the present invention, the hydrogel has a peak tack of at most about 1,000 grams, in other embodiments at most about 900 grams, and in yet other embodiments at most about 800 grams. In one or more embodiments of the present invention, the hydrogel has a peak tack of from about 15 grams to about 1,000 grams, in other embodiments from about 25 grams to about 900 grams, and in yet other embodiments from about 40 grams to about 800 grams.

In one or more embodiments of the present invention the hydrogel has a leg of at least about 0.2 mm, in other embodiments at least about 0.5 mm, and in yet other embodiments at least about 1.0 mm. In one or more embodiments of the present invention, the hydrogel has a leg of at most about 25 mm, in other embodiments at most about 23 mm, and in yet other embodiments at most about 21 mm. In one or more embodiments of the present invention, the hydrogel has a leg of from about 0.2 mm to about 25 mm, in other embodiments from about 0.5 mm to about 23 mm, and in yet other embodiments from about 1.0 mm to about 21 mm.

In one or more embodiments of the present invention the hydrogel has a work of adhesion of at least about 25 gram sec, in other embodiments at least about 75 gram sec, and in yet other embodiments at least about 100 gram sec. In one or more embodiments of the present invention the hydrogel has a work of adhesion of at most about 6,000 gram sec, in other embodiments at most about 5,800 gram sec, and in yet other embodiments at most about 5,600 gram sec. In one or more embodiments of the present invention the hydrogel has a work of adhesion of from about 25 gram sec to about 6,000 gram sec, in other embodiments from about 75 gram sec to about 5,800 gram sec, and in yet other embodiments from about 100 gram sec to about 5,600 gram sec.

In one or more embodiments of the present invention the hydrogel is selected form the group consisting of wherein the hydrogel material is selected from the group consisting of co-polyacrylate/acrylamide, glycerin, n-methyldiethanolamine, triethanolamine, potassium chloride, fumed silica, polyvinylpyrrolidone, water, and combinations thereof. In one or more embodiments of the present invention, the hydrogel comprises between about 25.0 to 35.0 weight % of co-polyacrylate/acrylamide, between about 15.0 to 50.0 weight % of glycerin, between about 0.0 to 6.0 weight % of n-methyldiethanolamine, between about 0.0 to 4.0 weight % of triethanolaminem between about 0.0 to 4.0 weight % of potassium chloride, between about 0.0 to 5.0 weight % of fumed silica, between about 0.0 to 2.0 weight % of polyvinylpyrrolidone, and between about 10.0 to 30.0 weight % water.

In one or more embodiments of the present invention, the hydrogel comprises a KM10T hydrogel manufactured by Katecho, Inc., Des Moines, Iowa. This hydrogel has a water absorption capacity of at most 1437%, a stainless steel adhesion of 735 grams per inch width, a pH of 6(±0.5), a thickness of 32 mils(±5), a volume resistivity of 1,055 Ohm-in, a peak tack of 210 grams, a leg of 4.7 mm, and a work of adhesion of 1,142 gram sec.

In one or more embodiments of the present invention, the bandage is more effective when applied to a wound site cleaned by mild soap and water prior to application of the bandage. In one or more embodiments of the present invention, the adhesion of the bandage may be reduced by the presence of dry skin or oil/lotion on the skin around the wound site. For example, the skin around the wound site should not be cleaned by alcohol or alcohol-based wipes prior to the application of the bandage as the alcohol will dry the skin.

In one or more embodiments of the present invention, the substrate used should be biocompatible with human use. In one or more embodiments of the present invention, the substrate is any tape or liner that adheres effectively to the hydrogel and padding material used such that the substrate selected forms a barrier for the bandage. In one or more embodiments of the present invention, the substrate for use in the bandage is medical tape. In one or more embodiments, the medical tape selected is a single coated polyurethane 9833 film manufactured by 3M Medical Specialties, St. Paul, Minn.

In one or more embodiments the padding material used in the bandage of the present invention should be is biocompatible with human use. In one or more embodiments of the present invention, the padding material used is a fiber material and can be a synthetic or natural material. The padding material is not critical in the use or manufacture of the bandage of the present invention and can be any known material such as a passive pad that absorbs fluids from the wound. In one or more embodiments, the padding material of the present invention can also contain active ingredients, such as antibiotics or anesthetics.

In one or more embodiments of the present invention, the padding material is selected from the group consisting of a hydrocolloid, cotton gauze, foam, a blend of lyocell and polyester, and combinations thereof. In one or more embodiments of the present invention, the padding material comprises a blend of 70% lyocell and 30% polyester.

In one or more embodiments of the present invention, the padding material used is 7.9NLYB-E padding material manufactured by SWM International, Alpharetta, Ga. The 7.9NLYB-E padding material is laminated with the Delnet® apertured non-adherent film, which has been found to be non-detrimental to the healing process of wounds.

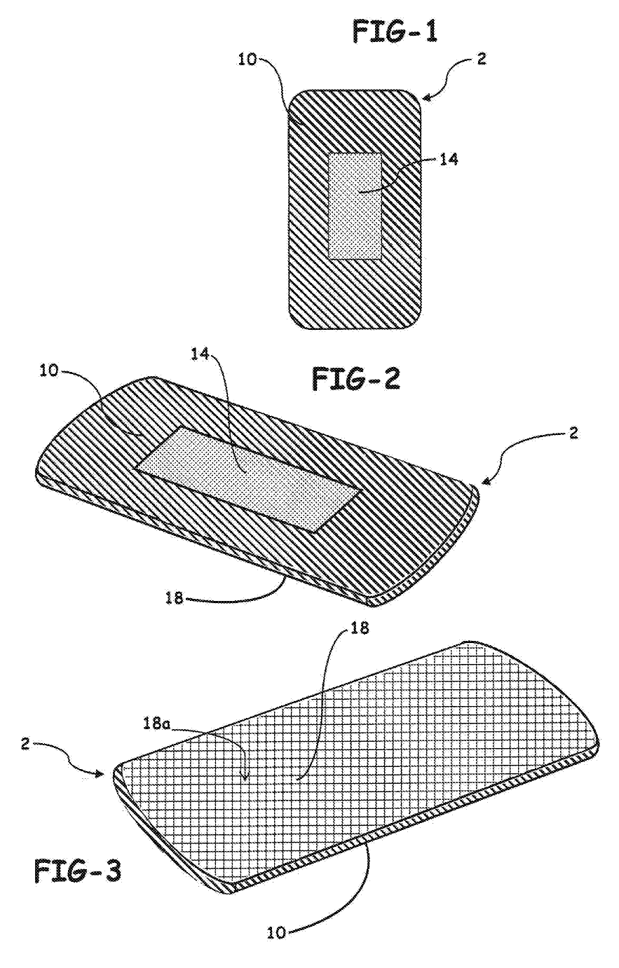

An embodiment of a bandage made in accordance with the present invention is generally indicated by the number 2. The shape and style of bandage 2 is discretionary and can take on the shape and style, for example, of a rectangular island pad style bandage 2, such as shown in FIG. 1. The chosen shape and style of bandage 2 is preferably one that will best provide coverage to a wound needing treatment and protection.

As shown in FIGS. 1-3, the bandage 2 of the present invention includes the following main components: a hydrogel 10, a padding material 14, and substrate 18. As shown in FIG. 1, hydrogel 10 surrounds padding material 14.

FIGS. 2, 3 show a rectangular island pad style bandage 2 and FIG. 5 shows a circular island pad style bandage 32. Both bandage 2 and bandage 32 include hydrogel 10 which surrounds padding material 14, both of which are adhered to an adhesive coated side of a substrate 18. The substrate 18 has a first planar surface 18a (shown in FIG. 3) and a second planar surface 18b (shown in FIG. 5).

Referring to FIG. 3, a perspective view of the top of a rectangular island pad style bandage 2 shows the rectangular island pad style bandage 2 further including a substrate 18.

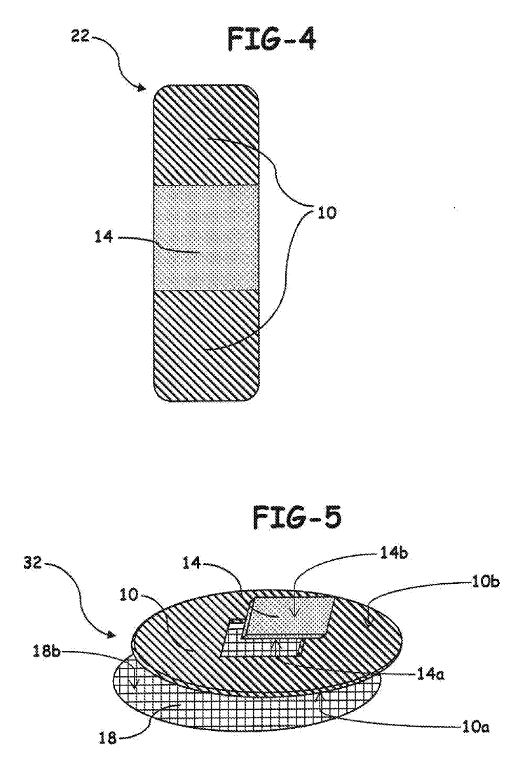

Referring to FIG. 4, illustrated is the bottom view of a traditional style bandage 22. Similar to the rectangular island pad style bandage 2, the traditional style bandage 2 comprises a hydrogel 10 and a padding material 14. In addition, although not shown in FIG. 4, hydrogel 10 and padding material 14 are both adhered to the adhesive coated side of a substrate 18.

Referring to FIG. 5, illustrated is an exploded perspective view of a circular island pad style bandage 32. Again, similarly to the rectangular island pad style bandage 2 and the traditional style bandage 22, the circular island pad style bandage 32 comprises a hydrogel 10, a padding material 14, and a substrate 18. The hydrogel 10 has a first planar surface 10a and a second planar surface 10b. The padding material 14 has a first planar surface 14a and a second planar surface 14b. Shown in the exploded view of FIG. 5 is the way in which the hydrogel 10 is sized to allow for co-disposal with the padding material 14. By use of the term co-disposal or co-disposed, it is meant that the hydrogel 10 and padding material 14 are situated such that both the first planar surface 10a of hydrogel 10 and the first planar surface 14a of padding material 14 are placed together on the second planar surface 18b of substrate 18.

In some embodiments, the bandage of the present invention comprises a padding material 14 with the hydrogel 10 only adjacent to a portion of padding material 14, in other embodiments, the bandage of the present invention comprises padding material 14 with hydrogel 10 adjacent to more than one portion of padding material 14 (FIG. 4), and in further embodiments the bandage of the present invention comprises padding material 14 with hydrogel 10 surrounding the entire padding material (FIGS. 1, 2, 5).

The composition of the padding material is not critical. In some embodiments of the present invention padding material 14 of the present invention is a non-woven material, in other embodiments padding material 14 of the present invention is a woven material, in other embodiments padding material 14 of the present invention is a foam material, and in further embodiments padding material 14 of the present invention is a plastic material. In one or more embodiment of the present invention, padding material 14 of the present invention is comprised of a different material than that of hydrogel 10.

The composition of substrate 18 is not critical. In some embodiments of the present invention, substrate 18 comprises a nonwoven sheet, in other embodiments, substrate 18 comprises a woven sheet, and in further embodiments, substrate 18 comprises a polymer film.

The present invention may also include a release liner which is disposed on the adhering side of bandage 2, opposite the position of substrate 18, to prevent contamination of hydrogel 10 and padding material 14 prior to use. Such release liners are well known in the art including those with a fluoropolymer coating to facilitate removal of the release liner.

To demonstrate the hypoallergenic property of the present invention, the following experiments were conducted. Six different bandages were tested on 8 human subjects, covering several different age groups, ethnic backgrounds, and included both male and female subjects. All subjects involved said they believed they had sensitive skin. The bandages compared were (1) the present invention in a circular shape, (2) 3M Nexcare™ waterproof bandage, (3) the present invention in a rectangular shape, (4) Curad™ Sensitive Skin bandage, (5) 3M Nexcare™ Tegaderm™ +Pad, and (6) CVS Gentle Strips. Bandages 2, 4, 5, and 6 are standard over the counter adhesive bandages. Bandage 2 is not described on its packaging as being hypoallergenic or for sensitive skin Bandage 4 is described on its packaging as being “protection for delicate skin” and is part of Curad's™ Sensitive Line of bandages. Bandages 5 and 6 are described on their packaging as being “hypoallergenic.”

The bandages were worn continuously for 24 hours. Each subject continued normal activities, including sleep, and were instructed not to intentionally expose the bandages to water. The bandages were applied to areas that are traditionally viewed as having sensitive skin. Each subject had bandages applied to the forearm, back of hand, and upper back. After 24 hours, the bandages were removed and the areas where the bandages were placed were observed to note the effects each bandage had on each subject's skin. In addition, each subject answered a survey indicating reactions based upon a scale of 0 to 3, with 0 being no reaction and 1-3 being rated as very little, somewhat, or severe. The survey included observations immediately after removal, 24 hours after removal, 72 hours after removal, and one week after removal.

The results of the comparison showed no undesirable traits for the hydrogel bandages in accordance with the present invention, whereas the other bandages from the comparison showed drawbacks included redness, discomfort, itchiness, hives and blisters. The worst bandages were those claiming to be hypoallergenic, and the best were the hydrogel bandages of the present invention that resulted in no negative effects. This supports the conclusion that a hydrogel based bandage serves as an effective medical device for hypoallergenic and sensitive skin wound care.

A bandage, made in accordance with the present invention, may be formed by applying each of the aforementioned components (hydrogel 10, padding material 14, and substrate 18) in the preferred structural configuration based on the desired shape and style of the product, by way of a multitude of procedures.

For example, one method of forming a bandage of the present invention contemplates individually forming elongated laminates of each component (hydrogel 10, padding material 14, and substrate 18) and then laminating those components in an order as to obtain the desired island pad style configuration, such as shown in FIGS. 1, 2, and 5. Padding material 14 would preferably be slit to a width to allow for co-disposal with the hydrogel 10 onto substrate 18. Hydrogel 10 would preferably be cut as to form cavities suitable for the aforementioned co-disposal with padding material 14 onto substrate 18. The aggregate laminate is formed by cutting padding material 14 and placing it on the adhesive coated side of substrate 18 followed by lamination of hydrogel 10 onto the adhesive coated side of substrate 18 and then laminating a release liner (not shown) with its fluoropolymer coated side adhered to the side of bandage 2 opposite the position of substrate 18. Thereafter, the aggregate laminate is cut or stamped into the desired shape and size for the bandage 2. In one or more embodiments of the present invention, the aggregate laminate can be formed by first laminating the hydrogel 10 onto the adhesive coated side of substrate 18 followed by lamination of the padding material 14.

What is claimed is:

1. A bandage comprising:
a. a padding material;
b. a hydrogel material; and
c. a substrate;
wherein the padding material is co-disposed with the hydrogel material and wherein both the padding material and the hydrogel material are carried by the substrate.

2. The bandage of claim 1 wherein the substrate has a first planar surface and a second planar surface and wherein the second planar surface is coated with an adhesive polymer.

3. The bandage of claim 2 wherein the padding material and the hydrogel material are carried by the substrate by means of the adhesive polymer coated on the second planar surface of the substrate.

4. The bandage of claim 3 wherein the padding material comprises a first planar surface and a second planar surface, wherein the hydrogel material comprises a first planar surface and as second planar surface, and wherein both the first planar surface of the hydrogel material and the first planar surface of the padding material are placed on the second planar surface of substrate.

5. The bandage of claim 4 wherein the substrate is a polyurethane film.

6. The bandage of claim 1 wherein the padding material is selected from the group consisting of a hydrocolloid, cotton gauze, foam, a blend of lyocell and polyester, and combinations thereof.

7. The bandage of claim 1 wherein the hydrogel material is selected from the group consisting of co-polyacrylate/acrylamide, glycerin, n-methyldiethanolamine, triethanolamine, potassium chloride, fumed silica, polyvinylpyrrolidone, water, and combinations thereof.

8. The bandage of claim 7 wherein the hydrogel material has a stainless-steel adhesion of from about 100 g/in² to about 2,000 g/in² and a pH of from about 4.0 to about 8.0.

9. The bandage of claim 7 wherein the hydrogel material has a water absorption capacity of from about 25% to about 3,500% and a thickness of from about 5 mils to about 70 mils.

10. The bandage of claim 1 further comprising a release liner adhered to the bandage at a position opposite a position of the substrate.

11. A process for creating a bandage comprising the steps of:
a. providing a substrate having a first planar surface and a second planar surface;
b. placing a hydrogel material onto the second planar surface of the substrate; and
c. placing a padding material onto the second planar surface of the substrate such that the padding material is co-disposed by the hydrogel material.

12. The process of claim 11 wherein the second planar surface of the substrate is coated with an adhesive polymer.

13. The process of claim 11 wherein the substrate comprises a polyurethane film.

14. The process of claim 10 wherein the padding material is selected from the group consisting of a hydrocolloid, cotton gauze, foam, a blend of lyocell and polyester, and combinations thereof.

15. The process of claim 11 wherein the hydrogel material is selected from the group consisting of co-polyacrylate/acrylamide, glycerin, n-methyldiethanolamine, triethanolamine, potassium chloride, fumed silica, polyvinylpyrrolidone, water, and combinations thereof.

16. The process of claim 15 wherein the hydrogel material has a stainless-steel adhesion of from about 100 g/in² to about 2,000 g/in² and a pH of from about 4.0 to about 8.0.

17. The process of claim 15 wherein the hydrogel material has a water absorption capacity of from about 25% to about 3,500% and a thickness of from about 5 mils to about 70 mils.

18. The process of claim 11 further comprising the step of placing a release liner on the bandage at a position opposite the substrate.
